(19) United States
(12) Patent Application Publication
(10) Pub. No.: US 2016/0278402 A1
(43) Pub. Date: Sep. 29, 2016

(54) Process for Producing Protein Concentrate and A Cellulosic Residue Material From Defatted Rice Bran

(71) Applicants: Ian MacKay, Eden Praire, MN (US); Karl Greden, Hinckley, MN (US)

(72) Inventors: Ian MacKay, Eden Praire, MN (US); Karl Greden, Hinckley, MN (US)

(21) Appl. No.: 15/171,780

(22) Filed: Jun. 2, 2016

Related U.S. Application Data
(63) Continuation-in-part of application No. 14/971,998, filed on Dec. 16, 2015.
Application No. 14/971,998 is a continuation-in-part of application No. 14/591,904, filed on Jan. 7, 2015.

(51) Int. Cl. A23K 10/14; A23K 50/10; C12P 19/14; C12P 21/06; C12P 19/04; A23K 10/38; A23K 50/75

(52) U.S. Cl. CPC A23K 10/14; A23K 10/38; A23K 50/10; A23K 50/75; C12P 21/06; C12P 19/04; C12P 19/14

(57) ABSTRACT

A process for treating defatted rice bran to produce a high value protein product and a cellulosic residue both from defatted rice bran. The high value protein product is useful as a protein supplement or feed for livestock and poultry and the cellulosic residue has value as a feedstock for a thermochemical process unit for the production of a biofuel. The defatted rice bran is subjected to both starch hydrolysis and protein hydrolysis and a resulting liquid stream containing hydrolyzed proteins is sent through a two membrane filtration stages, the first being a microfiltration and the second being a nanofiltration stage.

### CROSS-REFERENCE TO RELATED APPLICATIONS

This application is a Continuation-In-Part of application Ser. No. 14/971,998 filed Dec. 16, 2015, which is a Continuation-In-Part of Ser. No. 14/591,904 filed Jan. 7, 2015 which is based on Provisional Application 61/924,678 filed on Jan. 7, 2014.

### FIELD OF THE INVENTION

This invention relates to a process for treating defatted rice bran to produce a high value protein product and a cellulosic residue material. The high value protein product is useful as a food protein supplement or feed for livestock and poultry. The cellulosic residue product has value as a feedstock for a thermochemical process unit for the production of biofuel.

### BACKGROUND OF THE INVENTION

A substantial amount of research and development is being done to reduce our dependency on petroleum-based energy and to move us toward more sustainable and environmentally friendly energy sources, such as wind energy, solar energy, and energy derived from biomass. The conversion of biomass into transportation and other fuels is of great interest for reducing reliance on fossil fuels. Various biomass conversion technologies employ thermochemical processes, such as pyrolysis and gasification that have relatively high capital and operating costs. In particular, sourcing and preparing conventional biomass feedstocks, such as wood and agricultural residues, such as corn stover and soybean hulls, for pyrolysis or gasification, typically result in marginal production economics.

There is a need in the art for more economical and efficient processes for obtaining maximum value from waste biomass such as rice bran. Over 600 million tons of rice is harvested on a global scale. Much of the nutritional value of rice lies in the bran and germ, which consists mostly of the bran layer and germ of the rice with some fragments of hull and broken rice. Rice bran is typically comprised of protein, fat, carbohydrates and contains micro-nutrients such as vitamins, minerals, anti-oxidants and phytosterols. The high oil content of rice bran makes it subject to rancidification and is typically discarded during the milling process or used as low-value animal feed.

### SUMMARY OF THE INVENTION

In accordance with the present invention there is provided a process for producing a protein product and a cellulosic product suitable as a feedstock for thermochemical processing from defatted rice bran containing a starch component and a protein component, which process comprises:

a) introducing defatted rice bran into a hydrolysis reactor, along with an effective amount of water;

b) providing that the pH of the slurry be in the range from about 4.5 to about 6.5;

c) introducing an effective amount of a starch hydrolyzing enzyme into said hydrolysis reactor;

d) hydrolyzing at least a fraction of the starch of said defatted rice bran under hydrolysis conditions, including temperatures from about 10° C. to about 90° C. for an effective amount of time to result in a predetermined amount of starch to be converted to monosaccharides;

e) adjusting the pH of the slurry from about 10 to about 12;

f) introducing an effective amount of protease enzyme into said hydrolysis reactor and maintaining said hydrolyzing conditions for an effective amount of time to allow the degree of hydrolysis of proteins to reach about 12, thereby resulting in a slurry comprised of a liquid fraction containing hydrolyzed proteins, and monosaccharides, and other water solubles, and a solids fraction comprised of protein-lean cellulosic material;

g) conducting said slurry from step f) above to a liquid solids separation stage wherein said liquid fraction is separated from a wet-solids fraction;

h) conducting said liquid fraction to a membrane microfiltration stage containing a membrane capable of performing a separation in the micon size range thereby resulting in a retentate comprised of cellulosic residue material and a permeate comprised of water and hydrolyzed protein products and hydrolyzed starch products;

i) conducting said permeate to a membrane nanofiltration stage containing a membrane capable of performing a separation in the nano-size range, thereby resulting in a retentate comprised of hydrolyzed protein products, and a permeate comprised of an aqueous solution of water-soluble constituents, including monosaccharides, resulting from the above process steps;

j) spray drying said retentate resulting in a spray dried hydrolyzed protein product;

k) conducting said separated wet-solids fraction from step g) above and the retentate from step h) above to a drying zone to result in a dried cellulosic product; and

l) conducting said permeate from step i) above to a reverse osmosis stage wherein water-soluble constituents are removed and a recycle water stream is produced.

In a preferred embodiment, the defatted rice bran is pre-processed by milling it to a particle size of less than about 1 mm

Also in accordance with the present invention there is provided a process for producing a protein product and a cellulosic product suitable as a feedstock for thermochemical processing from defatted rice bran containing a starch component and a protein component, which process comprises:

a) introducing defatted rice into a hydrolysis reactor, along with an effective amount of water;

b) providing that the pH of the slurry is in the range of about 4.5 to about 6.5;

c) introducing an effective amount of a starch hydrolyzing enzyme into said hydrolysis reactor;

d) hydrolyzing at least a fraction of the starch of said defatted rice bran under hydrolysis including temperatures from about 10° C. to about 90° C. for an effective amount of time to result in a predetermined amount of starch to be converted to monosaccharides;

e) adjusting the pH of the slurry to a pH from about 9 to about 12 with an aqueous solution of a hydroxide of an alkali or alkaline earth metal;

f) maintaining said hydrolyzing conditions for an effective amount of time to allow the degree of hydrolysis of proteins to reach about 12, thereby resulting in a slurry comprised of a liquid fraction containing hydrolyzed proteins, and monosaccharides, and other water solubles, and a solids fraction comprised of protein-lean cellulosic material;

g) conducting said slurry from step f) above to a liquid solids separation stage wherein said liquid fraction is separated from said solids fraction;

h) conducting said liquid fraction to a membrane microfiltration stage containing a membrane capable of performing a separation in the micon size range thereby resulting in a retentate comprised of cellulosic residue material and a permeate comprised of water and hydrolyzed protein products and hydrolyzed starch products;

i) conducting said permeate to a membrane nanofiltration stage containing a membrane capable of performing a separation in the nano-size range, thereby resulting in a retentate comprised hydrolyzed protein products and a permeate comprised of an aqueous solution of water-soluble constituents, including monosaccharides, resulting from the above process steps;

j) spray drying said retentate resulting in a spray dried hydrolyzed protein product;

k) conducting said separated wet-solids fraction from step g) above and the retentate from step h) above to a drying zone to result in a dried cellulosic product; and

l) conducting said permeate from step i) above to a reverse osmosis stage wherein water-soluble constituents are removed and a recycle water stream is produced.

In a preferred embodiment, the metal hydroxide is selected from sodium and potassium hydroxide.

In a preferred embodiment, the milled defatted rice bran, after being treated with water, is subjected to an effective amount of ultrasonic energy capable of improving the accessibility of proteins of the defatted rice bran.

In another preferred embodiment the base is a mineral base preferably sodium hydroxide.

### BRIEF DESCRIPTION OF THE FIGURES

FIG. 1 hereof is a simplified flow scheme of one preferred embodiment of the process of the present invention for producing a protein rich product and a cellulosic residue material from defatted rice bran. This figure shows an optional stage (milling) for reducing the average particle size of the defatted rice bran in the event it is received having an average particle size too large for the instant process.

FIG. 2 hereof is simplified flow scheme of another preferred embodiment of the present invention showing both an optional milling stage and an optional sonication stage. Also, a base solution is used to hydrolyze proteins instead of a protease enzyme.

### DETAILED DESCRIPTION OF THE INVENTION

As previously mentioned, rice bran is a nutrient-dense by-product from the milling of rice. Unprocessed rice bran will typically be comprised of about 18 to 23 wt. % carbohydrates other than starch, about 18 to 30 wt. % starch, about 15 to 18 wt. % proteins, and about 18 to 23 wt. % fats (oils). Rice bran most suitable for the practice of the present invention is defatted rice bran wherein as much of the fat is removed, as possible, by any suitable method. One particular suitable method for removing fats from rice bran is solvent extraction, which can include supercritical solvent extraction. Solvent extraction is well known in the art. Preferred solvents include the C3 to C6 alkanes, more preferably propane and hexane. The term “defatted rice bran” as used herein means a rice bran that has gone through a defatting process, such as solvent extraction, and contains no more than about 3 wt. % fat, such as from about 0.1 to 3 wt. %, preferably from about 0.2 to about 3 wt. %, more preferably from about 0.5 to about 2.5 wt. %, and most preferably from about 0.5 to about 2.0 wt. % fat. These weight percents are based on the total weight of the rice bran excluding water.

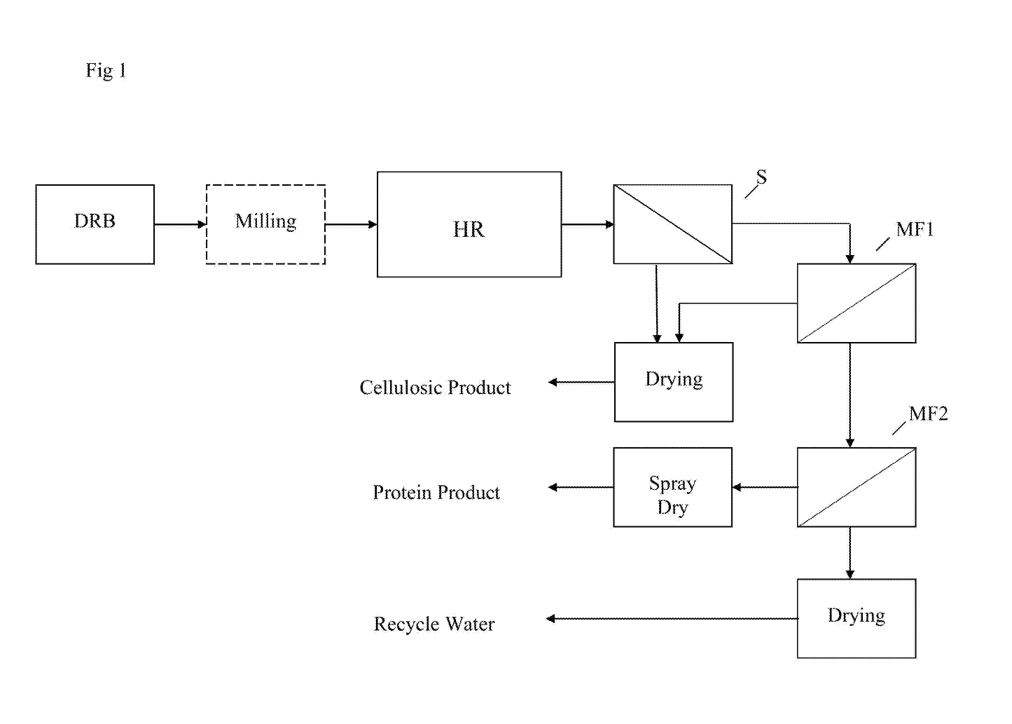

The present invention can be better understood with reference to the figures hereof. FIG. 1 hereof is one preferred embodiment wherein dry defatted rice bran DRB can be milled to reduce its average particle size if necessary. It is preferred, for purposes of the instant process, that the defatted rice bran have an average particle size from about 0.05 mm to about 1 mm, preferably from about 0.05 to about 0.5 mm, more preferably from about 0.05 to about 0.3 mm The defatted rice bran feed is then introduced into hydrolyzing reactor HR with an effective amount of water. By “effective amount of water” we mean at least that amount of water needed to make a slurry that can be efficiently mixed in a conventional stirred tank so that insoluble and soluble components stay in contact during hydrolysis. Such an effective amount of water will preferably be from about 9 to 1 to 10 to 1 water to defatted rice bran, on a weight basis. Two different types of hydrolysis reactions will be performed in hydrolysis reactor HR. One hydrolysis reaction is starch hydrolysis where the starch is hydrolyzed to monosaccharides, or sugars. The other is protein hydrolysis wherein long chain proteins are converted to shorter chain peptides and amino acids.

In the case of starch hydrolysis, the slurry will preferably be adjusted to be in the pH range of about 4.5 to 6.5, more preferably from about 5 to 6. Any suitable acid or base can be used to either raise or lower the pH to the desired range. If a base is needed it is preferred to use an aqueous solution of a metal hydroxide wherein the metal is selected from the alkali and alkaline earth metals. Preferred metals are sodium, potassium, calcium, and magnesium. More preferred are sodium and potassium, with sodium being the most preferred. The preferred acid is a mineral acid, more preferably hydrochloric acid. At least a portion of starch of the defatted rice bran is hydrolyzed by use of an effective amount of a starch hydrolyzing enzyme, preferably an amylase enzyme. By effective amount of starch hydrolyzing enzyme, we mean at least that amount needed to convert the starch, or apparent starch, content by about 80% to 99%, preferably from about 90% to 99% to monosaccharides. Any suitable amylase enzymes can be used in the practice of the present invention. Non-limiting examples of amylase enzymes that can be used in the practice of the present invention include fungal alpha-amylase, bacterial alpha-amylase, and fungal glucoamylase. Fungal glucoamylase enzymes are preferred. The amylase enzyme treated defatted rice bran is subjected to hydrolysis conditions to cause the starch and apparent starch to hydrolyze to monosaccharides, thus resulting in molecules small enough to be membrane separated from the hydrolyzed protein moieties extracted in the following step. The amylase enzyme will preferably be used as an aqueous solution of an effective concentration of about 0.1 to 1 wt. %, preferably from about 0.2 to 0.4 wt. %, based on the dry weight of the defatted rice bran.

Starch hydrolyzing, as well as protein hydrolyzing, conditions include temperatures from about 10° C. to about 90° C., preferably from about 20° C. to about 80° C., more preferably from about 30° C. to about 70° C. and most preferably from about 40° C. to about 60° C.; and times from about 10 minutes to 180 minutes, preferably from about 30 minutes to about 120 minutes, and more preferably from about 40 minutes to about 80 minutes. pH range for starch hydrolysis is from about 4.5 to 6.5 and the pH range for protein hydrolysis using a protease enzyme will be from about 10 to 12.

After a predetermined percent, preferably at least about 90%, of starch is hydrolyzed to monosaccharides the pH of the slurry is raised to about 10 to 12 with use of an aqueous base solution as previously discussed. An effective amount of a protease enzyme is added to hydrolyze at least a portion, preferably a major portion greater than 80%, more preferably greater than 90%, of the proteins of the defatted rice bran in the slurry. By effective amount of protease enzyme, we mean at least that amount needed to reduce at least about 5% to about 12%, preferably from about 9% to about 11%, of the average protein chain length in the defatted rice bran to smaller chain peptides and amino acids. Another way to measure an effective amount of protease enzyme is that minimum about that will result in a degree of protein hydrolysis of about 10 to 12, preferably 12. Any suitable protease enzyme can be used in the practice of the present invention. Non-limiting examples of protease enzymes that can be used in the practice of the present invention include serine proteases, threonine proteases, cysteine proteases, aspartate proteases, glutamic acid proteases, and metalloproteases. Aspartate and serine proteases are preferred, with serine being more preferred. The enzyme treated defatted rice bran are subjected to hydrolysis conditions to cause at least a fraction of the proteins of the defatted rice bran to hydrolyze, thus resulting in water soluble smaller chain constituents, such as peptides and amino acids. The protease enzyme will preferably be used in an aqueous solution at a concentration that will result in a predetermined level of protein hydrolysis, but will preferably be in the range of about 0.5 to 2 wt. %, more preferably from about 0.8 to 1.2 wt. %, based on the total dry weight of defatted rice bran being treated.

The resulting enzyme treated defatted rice bran slurry is conducted from reactor HR to a liquid/solids separation stage S resulting in a liquid fraction comprised of water, hydrolyzed proteins, hydrolyzed starch, and minor amounts of other water soluble constituents and a solids fraction comprised of the remaining defatted rice bran material, preferably a cellulosic residue material having a substantially reduced level of proteins. Non-limiting examples of other water soluble constituents include ash, salts, sugars, and dietary fibers. It is preferred that the separation stage include use of a centrifuge. The resulting separated solids can become part of solids stream which is sent to a drying stage, or it can be sent independently to a drying stage.

The resulting liquid fraction is further processed to isolate proteins from the other solubles by conducting the liquid fraction to first membrane filtration stage MF1 which preferably contains one or more membranes having pores in the microfiltration size range, typically from about 0.1 to about 10 micrometers (μm). The filtration will preferably be conducted using micrometer sized cylindrical through pores that pass through the membrane at a 90° angle. Membrane filtration is well known in the art, therefore no detailed discussion of it is necessary in this document. This first membrane filtration stage will contain one or more microfiltration membranes that will have a molecular weight cutoff of about 300 to 800 kDa (Daltons), preferably from about 400 to 600 kDa. Diafiltration is preferably used so that most of the protein is in the permeate. Diafiltration is well known in the art and typically uses ultrafiltration membranes to remove, or to lower, the concentration of salts or solvents from solutions containing proteins, peptides, nucleic acids, and other biomolecules. The retentate from membrane filtration stage MF1 is concentrated up to about 20% solids at the end of the filtration stage. The retentate will be comprised of fats, fibers, and possibly a small amount of unconverted proteins. The retentate can be added to the wet solids from separation stage S for drying, or sent independently to a drying stage.

The protein-rich permeate of this first membrane filtration stage, which will also contain other water soluble constituents, is conducted to second membrane filtration stage MF2 which contains a nanofiltration membrane to further purify and dewater the proteins. Second membrane filtration stage MF2 will have a molecular weight cutoff of about 250 to 2000 Daltons, preferably from about 500 to 1000 Daltons. Diafiltration is preferably used to demineralize the retentate of second membrane filtration stage MF2 as well as to remove sugars from the retentate. The permeate of second membrane filtration stage MF2 will be a low, if any, solids stream containing, inter alia, salts, ash, sugars, and relatively low molecular weight proteins, peptides and amino acids. The retentate will have a solids content of about 15 to 25 wt. %, preferably greater than about 20 wt. %, and will be comprised of the protein isolate. The resulting protein isolate solution is spray dried in spray drying stage SD resulting in a substantially dry protein product.

The permeate from membrane filtration stage MF2 is conducted to reverse osmosis stage RO wherein substantially all, that is at least about 95 wt. %, preferably at least about 98 wt. %, of the other water soluble constituents are removed to produce a recycle water RW.

The solids fraction, from both the separation stage and first membrane filtration stage MF1 are dried to result in a cellulosic residue product that is suitable as a feed source for both humans and livestock and as feedstock for a thermochemical process that can be converted into a transportation or other fuel.

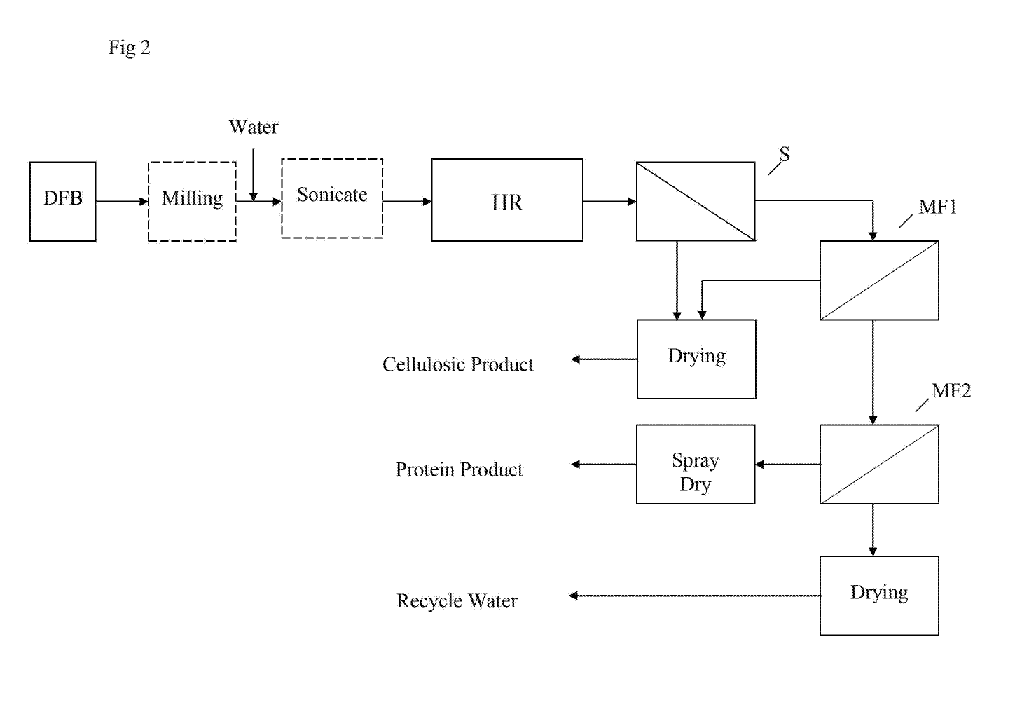

Reference is now made to FIG. 2 hereof which represents another preferred embodiment of the present invention for processing defatted rice bran to produce a protein concentrate or isolate product and a protein-lean residue (cellulosic) that can be used as a feed component for humans or livestock, or as feedstock for a thermochemical process to produce a biofuel. In this embodiment, the defatted rice bran is also optionally milled in the event it is received with too big a particle size to the average size range as discussed above for FIG. 1. An effective amount of water is added to the rice bran, preferably at a ratio of 9:1 to 10:1 water to dry bran and the resulting defatted rice bran slurry optionally subjected to sonication to help with protein removal. Ultrasonic energy helps to breakdown cell structures thereby improving access to proteins. The preferred effective ultrasonic energy input is from about 3 to about 30 Joules/gram of defatted rice bran with a frequency of about 40 kHz with about 3 to about 10 Joules/gram being preferred. It will be understood that ultrasonic energy can also be used in the process represented in FIG. 1 hereof.

A starch hydrolyzing enzyme can be added and subjected to hydrolysis conditions as was discussed with respect to FIG. 1 above.

After the predetermined level of starch hydrolysis is reached the pH is raised using a suitable amount of alkali or alkaline earth metal hydroxide solution to raise the pH to a range of about 9 to 12, preferably about 10 to about 12. Preferred metals of the hydroxide are sodium, potassium, magnesium and calcium, with sodium and potassium being the more preferred and sodium being the most preferred. A protease enzyme is not added in this embodiment, but protein hydrolysis proceeds by the action of the hydroxide solution at the aforementioned pH range.

By effective extraction conditions we mean extraction at a pH of about 9 to about 12, preferably at pH of about 10 to about 12; at a temperature range of about 30° C. to about 70° C., preferably from about 40° C. to about 60° C.; and with a bran to basic solution ratio of about 1:5 to about 1:10.

The resulting slurry that has undergone both starch hydrolysis and protein hydrolysis is conducted to a liquid/solids separation zone wherein a liquid fraction containing dissolved proteins and other water soluble constituents is separated from a predominantly solids fraction comprised of the remaining defatted rice bran having a substantially reduced level of proteins. It is preferred that the separation be done by centrifuge. The liquid fraction containing proteins is further purified in first membrane filtration stage MF1 using membrane filtration to remove non-protein molecules. The first membrane stage is a microfiltration membrane that will have a molecular weight cutoff of about 300 to 800 kDa and preferably from 400 to 600 kDa. Diafiltration is used so that most of the protein is in the permeate. The retentate is concentrated up to 20% solids at the end of the filtration process. The retentate contains fats, fibers, and a minimal amount of protein. The retentate can be added to the wet solids from the centrifuge for drying.

The protein rich permeate of first membrane filtration step MF1, which will also contain other water soluble constituents, is transferred to second membrane filtration stage MF2 which contains a nanofiltration membrane to further purify and dewater the proteins. Second membrane filtration stage MF2 will have a molecular weight cutoff of about 250 to 2000 Daltons, preferably from about 500 to 1000 Daltons. Diafiltration is used to demineralize the retentate as well as to remove sugars from the retentate. The permeate of second membrane filtration stage MF2 will be a low solids stream containing salts, ash, sugars, and low molecular weights proteins and amino acids. The retentate will have a solids content of about 15 to 25 wt. %, preferably greater than about 20 wt. %, that contains the protein isolate. The resulting protein isolate solution is spray dried in spray drying stage SD resulting in a substantially dry protein product.

The protein-lean cellulosic residue is collected and can be marketed as a livestock feed component, or as a feedstock component for a subsequent thermochemical process, such as pyrolysis or gasification, that can be used for the production of biofuel, preferably a transportation fuel, more preferably a distillate fuel. The protein product obtained by the practice of the present invention will be a protein concentrate comprised of at least 80 wt. % protein.

In a first preferred embodiment of the present invention the dry defatted rice bran is milled, either to less than 0.5 mm wherein an effective amount of water is added so that the water to bran ratio is about 10:1. The resulting mixture is heated to a temperature of about 50° C. and the pH of mixture is adjusted to a value of about 10.5 with use of a suitable base, preferably sodium hydroxide. The resulting solution is kept at this pH and temperature for about one hour wherein the pH is lowered to about 9 with use of a suitable acid, preferably hydrochloric acid. An effective amount of an alkaline protease, preferably alcalase, at a dosage of 10 mls/kg of protein is then added. The desired pH is maintained until a degree of hydrolysis of about 5 is reached, as measured by base addition.

In a second preferred embodiment of the present invention, the dry defatted rice bran, is milled to an average particle size of less than 0.5 mm wherein water is added until the water to bran ratio is about 10:1. The resulting mixture is then heated to a temperature of about 60° C. and the pH adjusted to about 9 with use of a suitable base material, preferably sodium hydroxide. An effective amount of an alkaline protease, such as alcalase, is then added at a dosage of 10 mls/kg of protein. The pH of 9 is maintained until a degree of hydrolysis of 12 is reached as measured by base addition.

In a third preferred embodiment of the present invention, the instant invention is performed by milling the dry defatted rice bran to less than 0.5 mm then adding water so that the water to grain ratio is 10:1. The resulting mixture is then heated to a temperature of about 50° C. and the pH adjusted to a value of about 11 using a suitable base material, preferably sodium hydroxide. The resulting solution is maintained at this pH and temperature for about 1 hr, then the pH is lowered to about 9 with use of a suitable acid, preferably hydrochloric acid.

In a fourth preferred embodiment and after doing any of treatments of the above first through third preferred embodiments, the pH is lowered to about 5 with use of a suitable acid material, preferably hydrochloric acid. The temperature is then adjusted to about 55° C. and an effective amount of a starch hydrolyzing enzyme, such as glucoamylase, is added to account for about 0.3% of total solids present. The pH and temperature is maintained for about 1 hr to hydrolyze the starch to glucose. The pH is then adjusted to a value of about 7.

In a fifth preferred embodiment the procedure of the above first through third embodiments is followed, but after the milling and water addition steps, the pH is adjusted to about 5 and an effective amount of glucoamylase is added as described in the above fourth preferred embodiment. After the 1 hr reaction period, the pH is adjusted to a value as described in the first through third preferred embodiment and the process continues as described in those embodiments.

In a sixth preferred embodiment the milling step in the first through third preferred embodiments is replaced with a hydrocavitation. Hydrocavitation is the process by which a fluid is passed through a small orifice to create controlled cavitation of the fluid resulting in localized high pressure and temperature. This process can disrupt and rupture cell bodies, opening up the cell structure and making it easier to solubilize the protein, or ultrasonic, treatment step wherein the defatted rice bran is subjected to the ultrasound waves for 120 seconds (range of 30 to 120 seconds) at a power density of 1 W/mL (range of 0.3 to 2.56 W/mL)

In a seventh preferred embodiment the procedure of the above first through third embodiments is followed, but after the milling and water addition steps an ultrasound step is conducted as described in the above sixth preferred embodiment.

In a more preferred embodiment of the present invention the dry defatted rice bran, is milled to less than 0.5 mm and an effective amount of water is added so that the water to bran ratio is 10:1. The resulting mixture is heated to about 55° C. and the pH of the resulting mixture is adjusted to a value of about 5 with use of a suitable acid or basic material, depending on the natural pH of the starting material. An effective amount of a starch hydrolyzing enzyme, preferably glucoamylase, is added at 0.3% of total solids present and the temperature and pH maintained for about 1 hr to hydrolyze the starch to glucose. The pH is adjusted to and the temperature raised to about 60° C. after 1 hr. An effective amount of a suitable alkaline protease, preferably alcalase, at a dosage of 10 mls/kg of protein is added. The pH and temperature are maintained until a degree of hydrolysis of about 12 is reached as measured by base addition. After a degree of hydrolysis of 12 is reached, the separation steps begin. Preferably glucoamylase is used to hydrolyze the starch to glucose. Fungal alpha amylase can be used to produce disaccharides and bacterial alpha amylase can be used for liquefaction of gelatinized starch prior to alpha amylase or glucoamylase treatment.

### EXAMPLE

Dry defatted rice bran is mixed with water so that the water to bran ratio is about 10:1. The mixture is then heated to 55° C. and hold at that temperature. The pH of the mixture is brought to a value of 5 with an appropriate base or acid such as sodium hydroxide or hydrochloric acid. A starch hydrolyzing amylase enzyme, such as glucoamylase, is added at 0.3% of total solids present. The temperature and pH is kept substantially constant for about 1 hr to hydrolyze the starch to glucose.

After 1 hr, the temperature of the mixture is raised up to about 60° C. and a pH to 9 using sodium hydroxide. An acceptable alkaline protease is added, such as alcalase, at a dosage of 10 mls/kg of protein. Keep at the desired pH until a degree of hydrolysis of 12 or higher is reached as measured by base addition.

What is claimed is:

1. A process for producing a protein product and a cellulosic product suitable as a feedstock for thermochemical processing from defatted rice bran containing a starch component and a protein component, which process comprises:
a) introducing defatted rice bran into a hydrolysis reactor, along with an effective amount of water;
b) providing that the pH of the slurry be in the range from about 4.5 to about 6.5;
c) introducing an effective amount of a starch hydrolyzing enzyme into said hydrolysis reactor;
d) hydrolyzing at least a fraction of the starch of said defatted rice bran under hydrolysis conditions, including temperatures from about 10° C. to about 90° C. for an effective amount of time to result in a predetermined amount of starch to be converted to monosaccharides;
e) adjusting the pH of the slurry to a pH from about 19 to about 12;
f) introducing an effective amount of protease enzyme into said hydrolysis reactor and maintaining said hydrolyzing conditions for an effective amount of time to allow the degree of hydrolysis of proteins to reach about 12, thereby resulting in a slurry comprised of a liquid fraction containing hydrolyzed proteins, and monosaccharides, and other water solubles, and a solids fraction comprised of protein-lean cellulosic material;
g) conducting said slurry from step f) above to a liquid solids separation stage wherein said liquid fraction is separated from a wet-solids fraction;
h) conducting said liquid fraction to a membrane microfiltration stage containing a membrane capable of performing a separation in the micon size range thereby resulting in a retentate comprised of cellulosic residue material and a permeate comprised of water and hydrolyzed protein products and hydrolyzed starch products;
i) conducting said permeate to a membrane nanofiltration stage containing a membrane capable of performing a separation in the nano-size range, thereby resulting in a retentate comprised of hydrolyzed protein products, and a permeate comprised of an aqueous solution of water-soluble constituents, including monosaccharides, resulting from the above process steps;
j) spray drying said retentate resulting in a spray dried hydrolyzed protein product;
k) conducting said separated wet-solids fraction from step g) above and the retentate from step h) above to a drying zone to result in a dried cellulosic product; and
l) conducting said permeate from step i) above to a reverse osmosis stage wherein water-soluble constituents are removed and a recycle water stream is produced.

2. The process of claim 1 wherein the average particle size of the defatted rice bran is from about 0.05 mm to about 1 mm

3. The process of claim 1 wherein the effective amount of water is about 10 to 1 water to defatted rice bran by weight.

4. The process of claim 1 wherein the pH of the slurry in step b) is from about 5 to about 6.

5. The process of claim 1 wherein the starch hydrolyzing enzyme is an amylase enzyme.

6. The process of claim 5 wherein the amylase enzyme is selected from the group consisting of fungal alpha-amylase, bacterial alpha-amylase, and fungal glucoamylase.

7. The process of claim 1 wherein the hydrolysis temperature is from about 30° C. to about 70° C.

8. The process of claim 1 wherein the protease enzyme is selected from the group consisting of serine proteases, threonine proteases, cysteine proteases, aspartate proteases, glutamic acid proteases, and metalloproteases.

9. The process of claim 1 wherein the feed defatted rice bran is first treated with an effective amount of water then subjected to ultrasonic energy prior to being treated with a hydrolyzing enzyme.

10. A process for producing a protein product and a cellulosic product suitable as a feedstock for thermochemical processing from defatted rice bran containing a starch component and a protein component, which process comprises:
a) introducing defatted rice into a hydrolysis reactor, along with an effective amount of water;
b) providing that the pH of the slurry is in the range of about 4.5 to about 6.5;
c) introducing an effective amount of a starch hydrolyzing enzyme into said hydrolysis reactor;
d) hydrolyzing at least a fraction of the starch of said defatted rice bran under hydrolysis including temperatures from about 10° C. to about 90° C. for an effective amount of time to result in a predetermined amount of starch to be converted to monosaccharides;
e) adjusting the pH of the slurry to a pH from about 9 to about 12 with an aqueous solution of a hydroxide of an alkali or alkaline earth metal;
f) maintaining said hydrolyzing conditions for an effective amount of time to allow the degree of hydrolysis of proteins to reach about 12, thereby resulting in a slurry comprised of a liquid fraction containing hydrolyzed proteins, and monosaccharides, and other water solubles, and a solids fraction comprised of protein-lean cellulosic material;
g) conducting said slurry from step f) above to a liquid solids separation stage wherein said liquid fraction is separated from said solids fraction;
h) conducting said liquid fraction to a membrane microfiltration stage containing a membrane capable of performing a separation in the micon size range thereby resulting in a retentate comprised of cellulosic residue material and a permeate comprised of water and hydrolyzed protein products and hydrolyzed starch products;
i) conducting said permeate to a membrane nanofiltration stage containing a membrane capable of performing a separation in the nano-size range, thereby resulting in a retentate comprised hydrolyzed protein products and a permeate comprised of an aqueous solution of water-soluble constituents, including monosaccharides, resulting from the above process steps;
j) spray drying said retentate resulting in a spray dried hydrolyzed protein product;
k) conducting said separated wet-solids fraction from step g) above and the retentate from step h) above to a drying zone to result in a dried cellulosic product; and
l) conducting said permeate from step i) above to a reverse osmosis stage wherein water-soluble constituents are removed and a recycle water stream is produced.

11. The process of claim 10 wherein the average particle size of the defatted rice bran is from about 0.05 mm to about 1 mm

12. The process of claim 10 wherein the effective amount of water is about 10 to 1 water to defatted rice bran by weight.

13. The process of claim 10 wherein the pH of the slurry in step b) is from about 5 to about 6.

14. The process of claim 10 wherein the starch hydrolyzing enzyme is an amylase enzyme.

15. The process of claim 14 wherein the amylase enzyme is selected from the group consisting of fungal alpha-amylase, bacterial alpha-amylase, and fungal glucoamylase.

16. The process of claim 10 wherein the hydrolysis temperature is from about 30° C. to about 70° C.

17. The process of claim 10 wherein the protease enzyme is selected from the group consisting of serine proteases, threonine proteases, cysteine proteases, aspartate proteases, glutamic acid proteases, and metalloproteases.

18. The process of claim 10 wherein the feed defatted rice bran is first treated with an effective amount of water then subjected to ultrasonic energy prior to being treated with a hydrolyzing enzyme.
